(19) United States
(12) Patent Application Publication
(10) Pub. No.: US 2016/0317231 A1
(43) Pub. Date: Nov. 3, 2016

(54) METHODS FOR MICROWAVE ABLATION PLANNING AND PROCEDURE USING A THREE-DIMENSIONAL MODEL OF A PATIENT

(71) Applicant: COVIDIEN LP, MANSFIELD, MA (US)

(72) Inventors: DARREN G. GIROTTO, LOUISVILLE, CO (US); JEETENDRA S. BHARADWAJ, LAFAYETTE, CO (US); KEVIN J. FRANK, LAFAYETTE, CO (US)

(21) Appl. No.: 15/099,772

(22) Filed: Apr. 15, 2016

Related U.S. Application Data
(60) Provisional application No. 62/154,924, filed on Apr. 30, 2015.
(60) Provisional application No. 62/154,929, filed on Apr. 30, 2015.
(60) Provisional application No. 62/154,933, filed on Apr. 30, 2015.
(60) Provisional application No. 62/154,942, filed on Apr. 30, 2015.
(60) Provisional application No. 62/154,950, filed on Apr. 30, 2015.
(60) Provisional application No. 62/154,958, filed on Apr. 30, 2015.

(51) Int. Cl. A61B 34/20; A61B 34/10; A61B 18/18

(52) U.S. Cl. CPC A61B 34/20; A61B 18/1815; A61B 34/10; A61B 2018/00577

(57) ABSTRACT

Disclosed are methods for microwave ablation planning and procedure using a three-dimensional model comprising displaying a three-dimensional model of at least a part of a patient's body generated based on image data acquired during imaging of the patient's body, displaying a pathway for navigating an ablation probe to at least one ablation target within the patient's body, tracking the location of the ablation probe inside the patient's body while the ablation probe is navigated along the pathway, displaying the tracked location of the ablation probe on the three-dimensional model, iteratively updating the displayed location of the ablation probe as the location of the ablation probe is tracked while the ablation probe is navigated inside the patient's body, and ablating the at least one target when the ablation probe is navigated proximate to the at least one target.

### CROSS-REFERENCE TO RELATED APPLICATION

The present application claims the benefit of and priority to U.S. Provisional Patent Application Ser. No. 62/154,942, filed on Apr. 30, 2015, by Darren G. Girotto, the entire contents of which is incorporated herein by reference.

### BACKGROUND

1. Technical Field

The present disclosure relates to systems, methods, and devices for planning and performing a microwave ablation treatment procedure.

2. Discussion of Related Art

When planning a treatment procedure, clinicians often rely on patient data including X-ray data, computed tomography (CT) scan data, magnetic resonance imaging (MRI) data, or other imaging data that allows the clinician to view the internal anatomy of a patient. The clinician utilizes the patient data to identify targets of interest and to develop strategies for accessing the targets of interest for the surgical procedure.

The use of CT images as a diagnostic tool has become routine and CT results are frequently the primary source of information available to a clinician regarding the size and location of a lesion, tumor, or other similar target of interest. This information is used by the clinician for planning an operative procedure such as a biopsy or an ablation procedure, but is only available as “offline” information that must typically be memorized to the best of the clinician's ability prior to beginning a procedure. During a CT scan, a patient is digitally imaged and a CT image data volume is assembled. The CT image data may then be viewed by the clinician each of the axial, coronal, and sagittal directions. A clinician reviews the CT image data slice by slice from each direction when attempting to identify or locate a target. It is often difficult, however, for the clinician to effectively plan a surgical ablation procedure based on the X-rays, CT images, or MRIs in their raw form.

### SUMMARY

Systems and methods for planning and performing a microwave ablation treatment procedure are provided.

According to an aspect of the present disclosure, a method comprises displaying a three-dimensional model of at least a part of a patient's body generated based on image data acquired during imaging of the patient's body, displaying a pathway for navigating an ablation probe to at least one ablation target within the patient's body, tracking the location of the ablation probe inside the patient's body while the ablation probe is navigated along the pathway, displaying the tracked location of the ablation probe on the three-dimensional model, iteratively updating the displayed location of the ablation probe as the location of the ablation probe is tracked while the ablation probe is navigated inside the patient's body, and ablating the at least one target when the ablation probe is navigated proximate to the at least one target.

In a further aspect of the present disclosure, the pathway to the at least one target is a straight line.

In another aspect of the present disclosure, the pathway extends between the at least one target and the exterior of the body of the patient.

In a further aspect of the present disclosure, the method further comprises tracking the location of at least one electromagnetic sensor located on the ablation probe.

In another aspect of the present disclosure, the method further comprises tracking the location of an ultrasound imager.

In another aspect of the present disclosure, the method further comprises displaying the tracked location of the ablation probe on real-time ultrasound images generated by the ultrasound imager.

In a further aspect of the present disclosure, the location of the ablation probe in relation to the at least one target is displayed on the real-time ultrasound images based on the tracked location of the ablation probe inside the patient's body.

In another aspect of the present disclosure, the location of the ablation probe in relation to the at least one target is displayed on the real-time ultrasound images based on the tracked location of the ultrasound imager.

In a further aspect of the present disclosure, the method further comprises displaying a model of a planned ablation zone in relation to the at least one target on the three-dimensional model.

In another aspect of the present disclosure, the method further comprises displaying a model of a planned ablation zone in relation to the at least one target on the real-time ultrasound images.

In a further aspect of the present disclosure, the method further comprises displaying, on the real-time ultrasound images, a projected ablation zone relative to the ablation probe.

In another aspect of the present disclosure, the method further comprises displaying, on the three-dimensional model, a projected ablation zone relative to the ablation probe.

In a further aspect of the present disclosure, the displayed location of the ablation probe is iteratively updated in relation to the pathway as the location of the ablation probe is tracked while the ablation probe is navigated inside the patient's body.

In another aspect of the present disclosure, the method further comprises displaying, on the three-dimensional model, a vector from the tip of the ablation probe indicating the trajectory of the ablation probe.

In a further aspect of the present disclosure, the method further comprises displaying, on the real-time ultrasound images, a vector from the tip of the ablation probe indicating the trajectory of the ablation probe.

In another aspect of the present disclosure, the method further comprises displaying, on the real-time ultrasound images, a shadow bar overlay indicating whether the trajectory of the ablation probe is in front of or behind the plane of the real-time ultrasound images.

In a further aspect of the present disclosure, the method further comprises inserting the ablation probe percutaneously into the patient's body.

Any of the above aspects and embodiments of the present disclosure may be combined without departing from the scope of the present disclosure.

### BRIEF DESCRIPTION OF THE DRAWINGS

Objects and features of the presently disclosed system and method will become apparent to those of ordinary skill in the art when descriptions of various embodiments thereof are read with reference to the accompanying drawings, of which:

FIG. 1 is a schematic diagram of a microwave ablation planning and procedure system in accordance with an illustrative embodiment of the present disclosure;

FIG. 2 is a schematic diagram of a computing device which forms part of the microwave ablation planning and procedure system of FIG. 1 in accordance with an embodiment of the present disclosure;

FIG. 3 is flow chart illustrating an example method of a procedure phase of a microwave ablation treatment in accordance with an embodiment of the present disclosure;

FIG. 4 is an illustration of a user interface presenting a view showing a setup step of the procedure phase of the microwave ablation treatment in accordance with an embodiment of the present disclosure;

FIG. 5 is an illustration of a user interface presenting a view showing a guidance step of the procedure phase of the microwave ablation treatment in accordance with an embodiment of the present disclosure;

FIG. 6 is an illustration of a user interface presenting a view showing guidance during the procedure phase of the microwave ablation treatment in accordance with an embodiment of the present disclosure; and

FIG. 7 is an illustration of a user interface presenting a view showing an ablation step of the procedure phase of the microwave ablation treatment in accordance with an embodiment of the present disclosure; and

FIG. 8 is another flow chart illustrating an example method of a procedure phase of a microwave ablation treatment in accordance with an embodiment of the present disclosure.

### DETAILED DESCRIPTION

The present disclosure provides a system and method for planning and performing microwave ablation surgical treatment. The system presents a clinician with a streamlined method of treatment planning from the initial patient selection through a process of target identification and selection, target sizing, treatment zone sizing, entry point and route selection to create a pathway to the target, and treatment plan review. The treatment plan may then be used as a guide during the performance of the surgical procedure, where the system is configured to track the position of surgical tools inside the patient and give the clinician a real-time view of the position of the tools in relation to the target and the pre-planned pathway toward the target. The system also presents a clinician with the capability to compare and contrast pre-operative and post-operative CT image data to assess the outcome of a surgical treatment procedure that has been performed.

Although the present disclosure will be described in terms of specific illustrative embodiments, it will be readily apparent to those skilled in the art that various modifications, rearrangements, and substitutions may be made without departing from the spirit of the present disclosure. The scope of the present disclosure is defined by the claims appended hereto.

Microwave ablation treatment, according to the present disclosure, is generally divided into two phases: (1) a planning phase, and (2) a procedure phase. The planning phase of microwave ablation treatment is more fully described in co-pending provisional patent application No. 62/035,851 entitled TREATMENT PROCEDURE PLANNING SYSTEM AND METHOD, filed on Aug. 11, 2014 by Bharadwaj et al., the contents of which is hereby incorporated by reference in its entirety. The alternative planning and a procedure phase are more fully described below.

A microwave ablation planning and procedure system according to the present disclosure may be a unitary system configured to perform both the planning phase and the procedure phase, or the system may include separate devices and software programs for the various phases. An example of the latter may be a system wherein a first computing device with one or more specialized software programs is used during the planning phase, and a second computing device with one or more specialized software programs may import data from the first computing device to be used during the procedure phase.

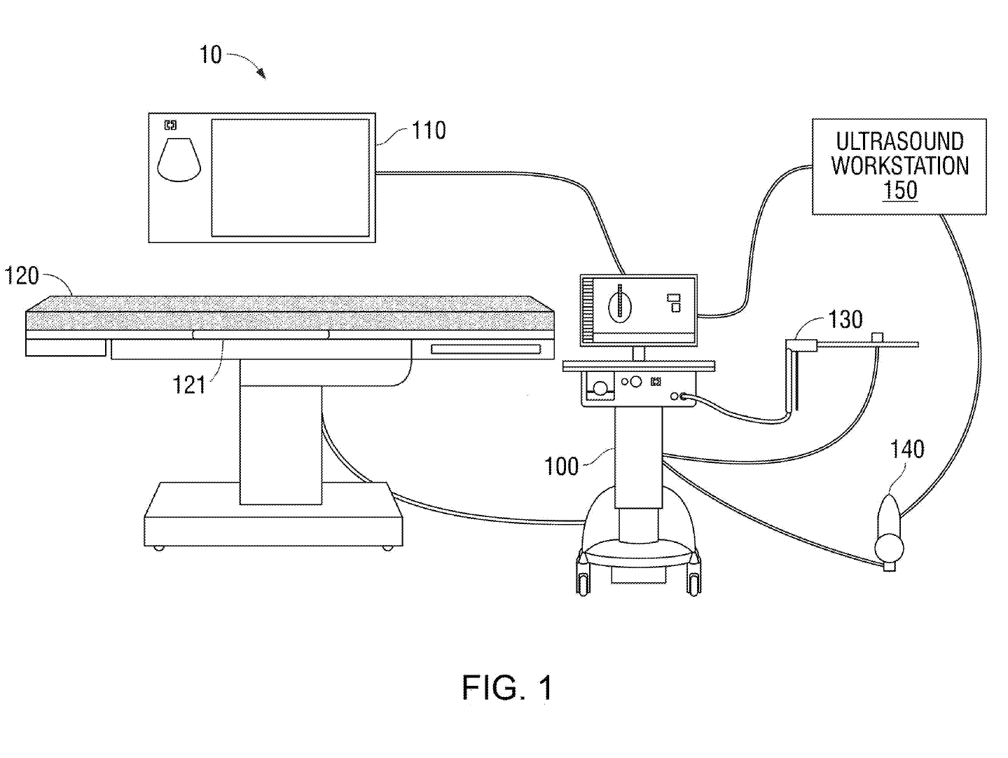

Referring now to FIG. 1, the present disclosure is generally directed to a treatment system 10, which includes a computing device 100, a display 110, a table 120, an ablation probe 130, and an ultrasound sensor 140 connected to an ultrasound workstation 150. Computing device 100 may be, for example, a laptop computer, desktop computer, tablet computer, or other similar device. Computing device 100 may be configured to control an electrosurgical generator, a peristaltic pump, a power supply, and/or any other accessories and peripheral devices relating to, or forming part of, system 10. Display 110 is configured to output instructions, images, and messages relating to the performance of the microwave ablation procedure. Table 120 may be, for example, an operating table or other table suitable for use during a surgical procedure, which includes an electromagnetic (EM) field generator 121. EM field generator 121 is used to generate an EM field during the microwave ablation procedure and forms part of an EM tracking system that is used to track the positions of surgical instruments within the body of a patient. EM field generator 121 may include various components, such as a specially designed pad to be placed under, or integrated into, an operating table or patient bed. An example of such an EM tracking system is the AURORA™ system sold by Northern Digital Inc. Ablation probe 130 is a surgical instrument having a microwave ablation antenna that is used to ablate tissue. While the present disclosure describes the use of system 10 in a surgical environment, it is also envisioned that some or all of the components of system 10 may be used in alternative settings, for example, an imaging laboratory and/or an office setting.

In addition to the EM tracking system, the surgical instruments may also be visualized by using ultrasound imaging. Ultrasound sensor 140, such as an ultrasound wand, may be used to image the patient's body during the microwave ablation procedure to visualize the location of the surgical instruments, such as ablation probe 130, inside the patient's body. Ultrasound sensor 140 may have an EM tracking sensor embedded within or attached to the ultrasound wand, for example, a clip-on sensor or a sticker sensor. As described further below, ultrasound sensor 140 may be positioned in relation to ablation probe 130 such that ablation probe 130 is at an angle to the ultrasound image plane, thereby enabling the clinician to visualize the spatial relationship of ablation probe 130 with the ultrasound image plane and with objects being imaged. Further, the EM tracking system may also track the location of ultrasound sensor 140. In some embodiments, one or more ultrasound sensors 140 may be placed inside the body of the patient. EM tracking system may then track the location of such ultrasound sensors 140 and ablation probe 130 inside the body of the patient. Ultrasound workstation 150 may be used to configure, operate, and view images captured by ultrasound sensor 140.

Various other surgical instruments or surgical tools, such as LigaSure™ devices, surgical staples, etc., may also be used during the performance of a microwave ablation treatment procedure. Ablation probe 130 is used to ablate a lesion or tumor (hereinafter referred to as a “target”) by using electromagnetic radiation or microwave energy to heat tissue in order to denature or kill cancerous cells. The construction and use of a system including such an ablation probe 130 is more fully described in co-pending provisional patent application No. 62/041,773 entitled MICROWAVE ABLATION SYSTEM, filed on Aug. 26, 2014, by Dickhans, co-pending patent application No. 13/836,203 entitled MICROWAVE ABLATION CATHETER AND METHOD OF UTILIZING THE SAME, filed on Mar. 15, 2013, by Latkow et al., and co-pending patent application No. 13/834,581 entitled MICROWAVE ENERGY-DELIVERY DEVICE AND SYSTEM, filed on Mar. 15, 2013, by Brannan et al., the contents of all of which is hereby incorporated by reference in its entirety.

The location of ablation probe 130 within the body of the patient may be tracked during the surgical procedure. An example method of tracking the location of ablation probe 130 is by using the EM tracking system, which tracks the location of ablation probe 130 by tracking sensors attached to or incorporated in ablation probe 130. Various types of sensors may be used, such as a printed sensor, the construction and use of which is more fully described in co-pending provision patent application No. 62/095,563 filed Dec. 22, 2014, the entire contents of which is incorporated herein by reference. Prior to starting the procedure, the clinician is able to verify the accuracy of the tracking system.

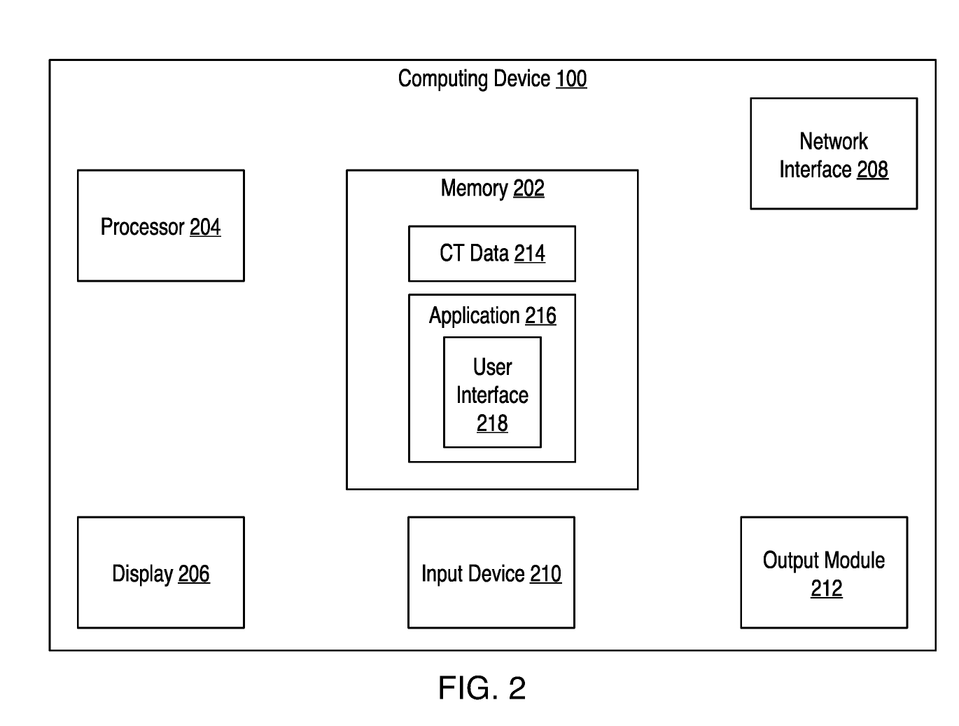

Turning now to FIG. 2, there is shown a system diagram of computing device 100. Computing device 100 may include memory 202, processor 204, display 206, network interface 208, input device 210, and/or output module 212.

Memory 202 includes any non-transitory computer-readable storage media for storing data and/or software that is executable by processor 204 and which controls the operation of computing device 100. In an embodiment, memory 202 may include one or more solid-state storage devices such as flash memory chips. Alternatively or in addition to the one or more solid-state storage devices, memory 202 may include one or more mass storage devices connected to the processor 204 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by computing device 100.

Memory 202 may store application 216 and/or CT data 214. Application 216 may, when executed by processor 204, cause display 206 to present user interface 218.

Processor 204 may be a general-purpose processor, a specialized graphics processing unit (GPU) configured to perform specific graphics processing tasks while freeing up the general-purpose processor to perform other tasks, and/or any number or combination of such processors.

Display 206 may be touch sensitive and/or voice activated, enabling display 206 to serve as both an input and output device. Alternatively, a keyboard (not shown), mouse (not shown), or other data input devices may be employed.

Network interface 208 may be configured to connect to a network such as a local area network (LAN) consisting of a wired network and/or a wireless network, a wide area network (WAN), a wireless mobile network, a Bluetooth network, and/or the internet. For example, computing device 100 may receive computed tomographic (CT) image data of a patient from a server, for example, a hospital server, internet server, or other similar servers, for use during surgical ablation planning. Patient CT image data may also be provided to computing device 100 via a removable memory 202. Computing device 100 may receive updates to its software, for example, application 216, via network interface 208. Computing device 100 may also display notifications on display 206 that a software update is available.

Input device 210 may be any device by means of which a user may interact with computing device 100, such as, for example, a mouse, keyboard, foot pedal, touch screen, and/or voice interface.

Output module 212 may include any connectivity port or bus, such as, for example, parallel ports, serial ports, universal serial busses (USB), or any other similar connectivity port known to those skilled in the art.

Application 216 may be one or more software programs stored in memory 202 and executed by processor 204 of computing device 100. As will be described in more detail below, during the planning phase, application 216 guides a clinician through a series of steps to identify a target, size the target, size a treatment zone, and/or determine an access route to the target for later use during the procedure phase. In some embodiments, application 216 is loaded on computing devices in an operating room or other facility where surgical procedures are performed, and is used as a plan or map to guide a clinician performing a surgical procedure, but without any feedback from ablation probe 130 used in the procedure to indicate where ablation probe 130 is located in relation to the plan. In other embodiments, system 10 provides computing device 100 with data regarding the location of ablation probe 130 within the body of the patient, such as by EM tracking, which application 216 may then use to indicate on the plan where ablation probe 130 are located.

Application 216 may be installed directly on computing device 100, or may be installed on another computer, for example, a central server, and opened on computing device 100 via network interface 208. Application 216 may run natively on computing device 100, as a web-based application, or any other format known to those skilled in the art. In some embodiments, application 216 will be a single software program having all of the features and functionality described in the present disclosure. In other embodiments, application 216 may be two or more distinct software programs providing various parts of these features and functionality. For example, application 216 may include one software program for use during the planning phase, and a second software program for use during the procedure phase of the microwave ablation treatment. In such instances, the various software programs forming part of application 216 may be enabled to communicate with each other and/or import and export various settings and parameters relating to the microwave ablation treatment and/or the patient to share information. For example, a treatment plan and any of its components generated by one software program during the planning phase may be stored and exported to be used by a second software program during the procedure phase.

Application 216 communicates with a user interface 218 that generates a user interface for presenting visual interactive features to a clinician, for example, on display 206 and for receiving clinician input, for example, via a user input device. For example, user interface 218 may generate a graphical user interface (GUI) and output the GUI to display 206 for viewing by a clinician.

Computing device 100 is linked to display 110, thus enabling computing device 100 to control the output on display 110 along with the output on display 206. Computing device 100 may control display 110 to display output which is the same as or similar to the output displayed on display 206. For example, the output on display 206 may be mirrored on display 100. Alternatively, computing device 100 may control display 110 to display different output from that displayed on display 206. For example, display 110 may be controlled to display guidance images and information during the microwave ablation procedure, while display 206 is controlled to display other output, such as configuration or status information.

As used herein, the term “clinician” refers to any medical professional (i.e., doctor, surgeon, nurse, or the like) or other user of the treatment planning system 10 involved in planning, performing, monitoring, and/or supervising a medical procedure involving the use of the embodiments described herein.

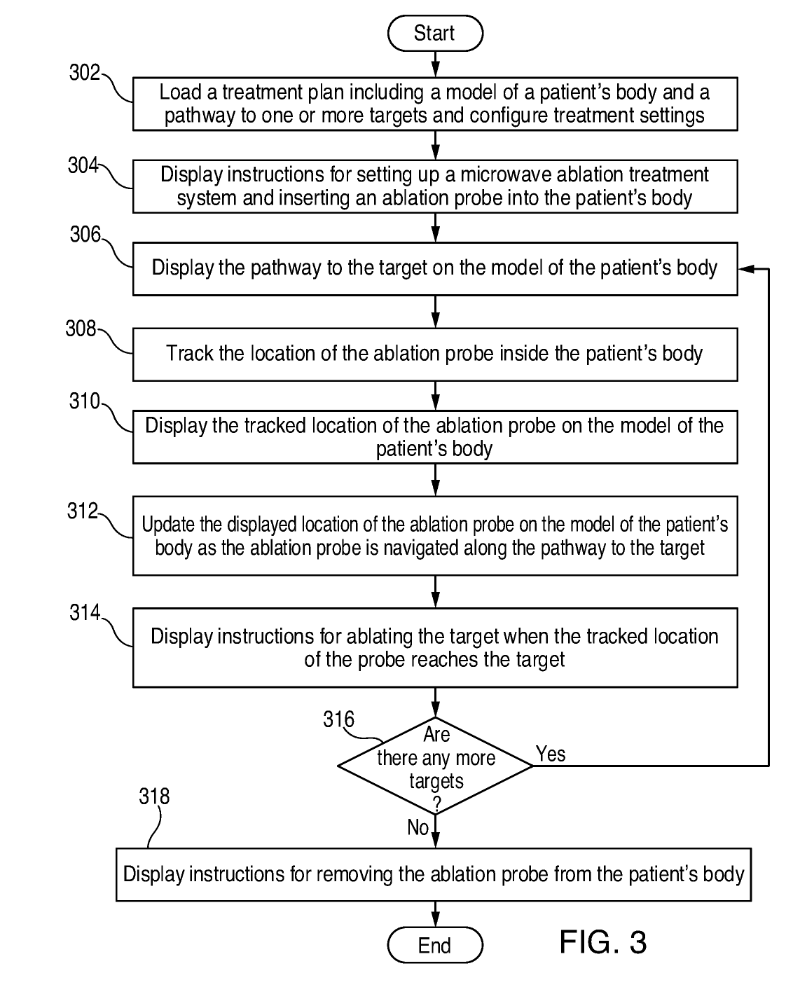

Turning now to FIG. 3, there is shown a flowchart of an example method for performing a microwave ablation procedure according to an embodiment of the present disclosure. At step 302, a clinician may use computing device 100 to load a treatment plan into application 216. The treatment plan may include a model of a patient's body and a pathway to one or more targets.

The model and treatment plan are both generated during the planning phase. The model may be generated based on CT image data acquired during a CT scan of the patient, although other imaging modalities are also envisioned. The clinician uses the model to select one or more targets for treatment during the microwave ablation procedure. Thereafter, application 216 generates a pathway from each selected target to an entry point on the patient's body where an ablation probe 130 may be inserted. The pathway is generated in such a way as to avoid any bones, vital organs, or other critical structures inside the patient's body. After loading the treatment plan on computing device 100, the clinician may view and modify the treatment plan.

The clinician may further configure the system settings for the microwave ablation procedure. For example, the clinician may preconfigure parameters related to the various tools to be used during the procedure, such as preconfiguring the output settings of ablation probe 130 for each target in the treatment plan. By doing so, application 216 may automatically configure a different output of ablation probe 130 when ablation probe 130 reaches each target.

The clinician may also view images or “snapshots” that were stored during the planning phase. For example, the clinician may store various images during the planning phase showing the targets from different angles. As noted above, the planning phase of microwave ablation treatment is more fully described in co-pending provisional patent application No. 62/035,851 entitled TREATMENT PROCEDURE PLANNING SYSTEM AND METHOD.

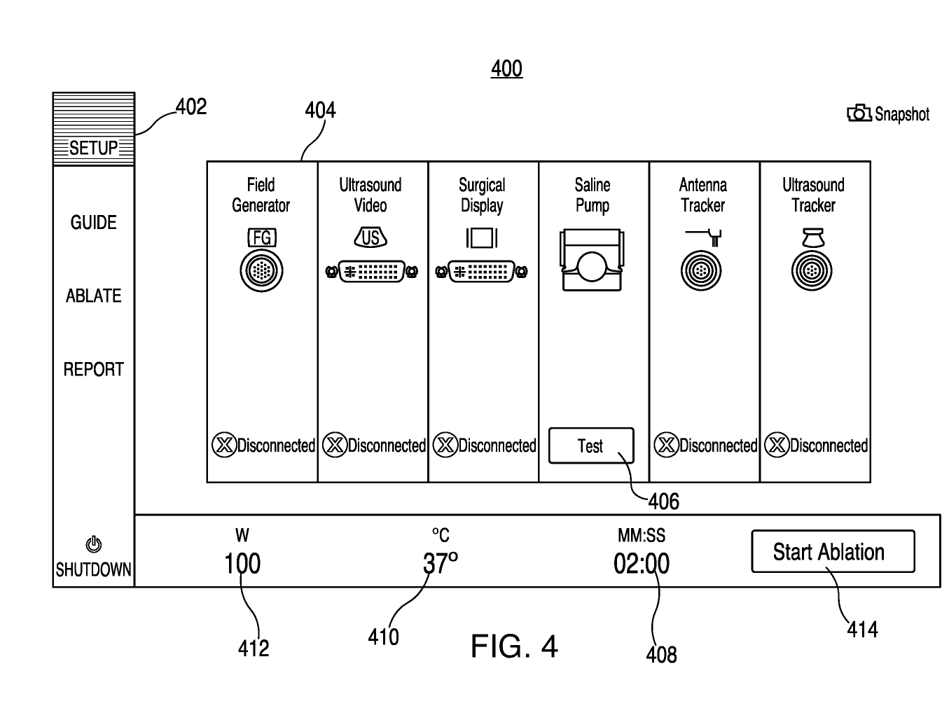

Then, at step 304, application 216, via user interface 218, displays instructions for setting up and configuring the microwave ablation system. The instructions may be visual and/or audible, and may provide feedback for proper versus improper system configuration. For example, as shown in FIG. 4, computing device 100 may display a system configuration screen 400. Screen 400 shows an indicator 402 of the step of ablation procedure in which the system is currently operating. Screen 400 further shows a list 404 that indicates various system components that should be connected for the procedure, as well as the status of those components. A button 406 is provided when a system component is connected to test the functioning of that component. Screen 400 also shows indicators representing the configured time 408, temperature 410, and output power 412 of ablation probe 130.

When the system has been configured for the procedure, the clinician may start the procedure, stop the procedure, pause the procedure, resume the procedure, and/or reset the procedure by selecting a button 414. Upon selecting button 414, application 216 causes computing device 100 to automatically start one or more of the system components. For example, application 216 may automatically start a peristaltic pump, an electrosurgical generator, and/or a power supply. Then, application 216 displays instructions for inserting ablation probe 130 into the patient's body. Thereafter, at step 306, application 216 displays the model of the patient's body with the pathway to the target as was generated in the planning phase.

In one embodiment, the treatment phase is similar to that employed by the iLogic® system currently sold by Covidien LP, in which the position of the patient in the magnetic field is registered with the images from the planning phase. In addition, the location of the ablation probe in the electromagnetic field is detected and displayed with reference to the planned pathway and the position of the patient and more specifically with respect to the target identified and displayed in the model.

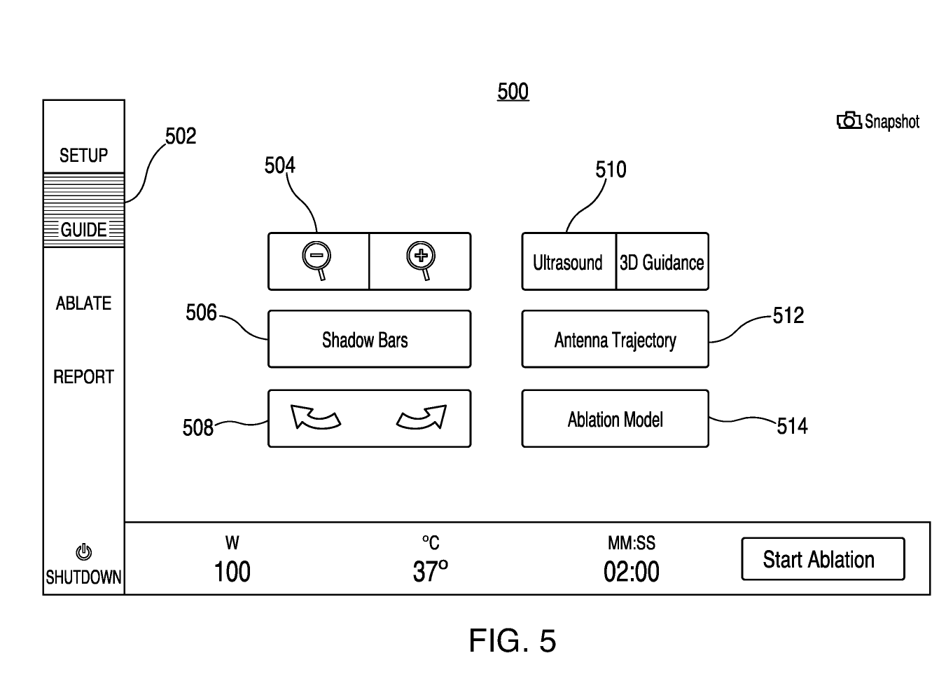

In an alternative or additional embodiment, the clinician navigates ablation probe 130 along the pathway to the target utilizing the ultrasound imaging system including ultrasound sensor 140 and ultrasound workstation 150. The navigation instructions, such as the pathway and other relevant information, may be displayed on display 110, while display 206 displays a configuration screen 500, as shown in FIG. 5, described below. While ablation probe 130 is navigated, application 216, at step 308, tracks the location of ablation probe 130 inside the patient's body, and, at step 310, displays the tracked location of ablation probe 130 on the model of the patient's body. In addition, the application 216 projects a vector extending from the end of the ablation probe 130 to give an indication to the clinician of the intersecting tissue along the trajectory of the ablation probe 130. In this manner, the clinician can alter the approach to a lesion or tumor to optimize the placement with a minimum of trauma.

Application 216, at step 312, iteratively updates the displayed location of ablation probe 130 on the model of the patient's body as ablation probe 130 is navigated along the pathway to the target.

When application 216 or the clinician detects that ablation probe 130 has reached the target, application 216, at step 314, displays instructions for ablating the target, including the settings previously set by the clinician for ablating the tumor, and enables the clinician to select the “start ablation” button to treat the target. When the “start ablation” button is selected, system 10 may automatically start other related accessories and/or peripheral devices, such as an associated peristaltic pump. Thereafter, at step 316, application 216 determines if there are any more targets in the treatment plan that have yet to be treated based on the planned procedure. If the determination is yes, the process returns to step 306 where the displayed pathway is updated to reflect the pathway to the next target. If the determination is no, application 216, at step 318, displays instructions for removing ablation probe 130 from the patient's body. During the ablation procedure, data relating to power and time settings as well as temperature data of ablation probe 130 for each ablation is continually stored.

Additionally, application 216 may present the clinician with instructions, such as a workflow, relating to protocols associated with a particular type of ablation procedure. For example, application 216 may present different workflows depending on the type of ablation procedure being performed, such that each of a track ablation, large tumor ablation, ablation of multiple tumors along a single track, and/or any other relevant ablation procedure may have instructions particularly tailored to the procedure.

Referring now to FIG. 5, there is shown an example screen 500 which may be displayed on display 206 either during the guidance step of the microwave ablation procedure or selected at any time by the clinician to adjust the features of the system 500. Screen 500 shows an indicator 502 that the system is now operating in the guidance step. Screen 500 further provides buttons 504 allowing the clinician to zoom in and out on the model and pathway displayed on display 110. Screen 500 further provides a button 506 that enables a shadow bar overlay on the pathway displayed on display 110 that indicates whether the trajectory of ablation probe 130 is in front of or behind an ultrasound image plane within the guidance view displayed on display 110. This enables the clinician to visualize the projected trajectory of ablation probe 130, as well as the interaction of the trajectory of ablation probe 130 within, or related to, the ultrasound image plane.

Screen 500 also includes buttons 508 allowing the clinician to rotate the guidance view displayed on display 110. Screen 500 further includes a button 510 allowing the clinician to toggle between a view of the model with the pathway and a live ultrasound image video feed. Screen 500 also includes a button 512 allowing the clinician to toggle the display of the planned pathway of ablation probe 130 on the model, and a button 514 allowing the clinician to toggle the display of a projected ablation zone relative to ablation probe 130 on the model to enable the clinician to visualize the ablation zone relative to ablation probe 130. The ablation zone may also be overlaid on the ultrasound images, thereby allowing the clinician to visualize the ablation zone within the ultrasound plane. The ablation zone may be presented to the clinician in a 2D and 3D ablation zone model.

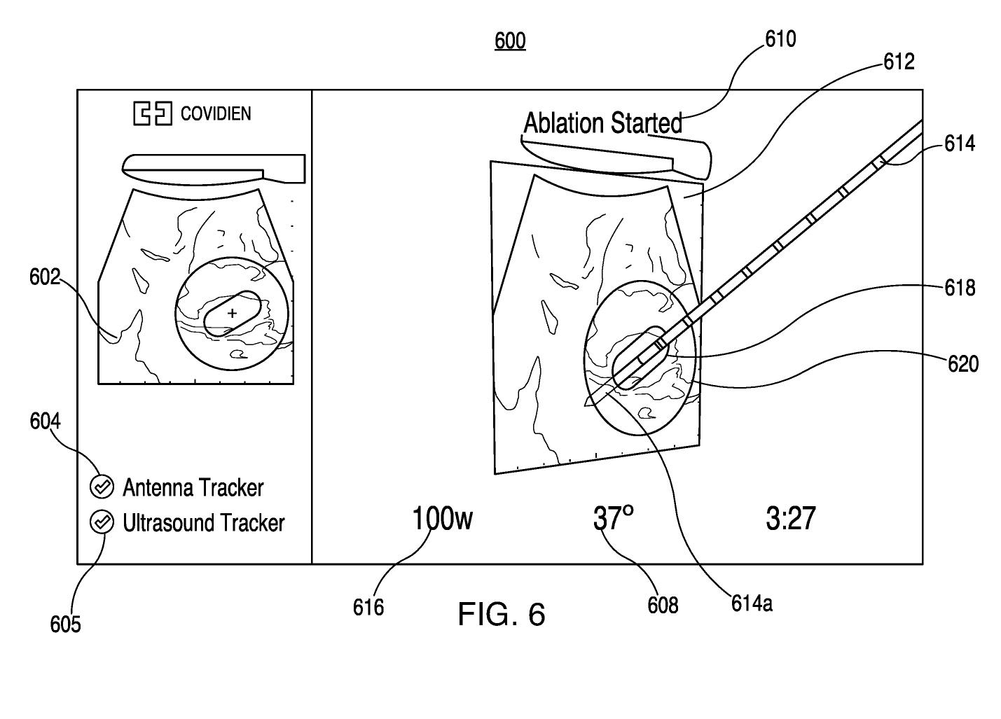

FIG. 6 shows an example screen 600 that may be displayed on display 110 during the microwave ablation procedure. Screen 600 includes a view 602 of the live 2D ultrasound images captured during the procedure. Screen 600 further shows a status indicator 604 for ablation probe 130 and a status indicator 606 for ultrasound sensor 140. Screen 600 also includes a view 608 for displaying status messages relating to the ablation procedure, such as a power setting of ablation probe 130, duration of the ablation and/or a time remaining until the ablation procedure is complete, progression of the ablation, feedback from a temperature sensor, and a zone chart used during the ablation procedure. Screen 600 further includes a view 610 for showing transient messages relating to the ablation procedure, such as changes caused by selecting the buttons provided by screen 500, described above. Screen 600 also displays the navigation view 612, which includes a representation 614 of ablation probe 130 as well as a shadow indicator 614a representing the portion of ablation probe 130 which lies below the ultrasound imaging plane, a vector line 616 representing the trajectory of the ablation probe, a current ablation zone 618 showing the area which is currently being ablated, and a total ablation zone 620 showing the area which will be ablated if the ablation procedure is allowed to run to completion.

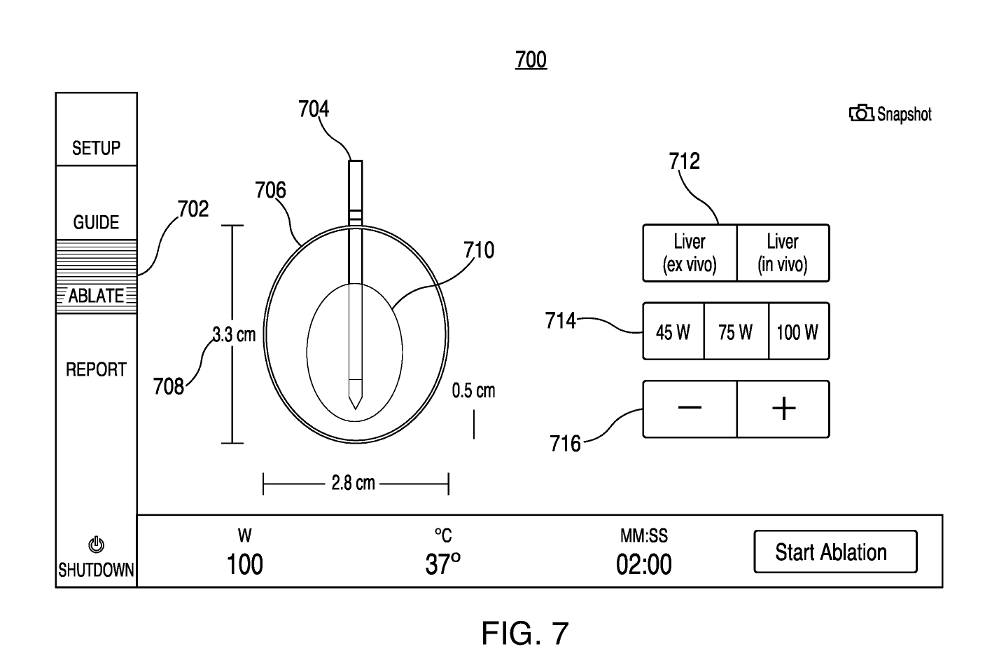

FIG. 7 shows an example screen 700 that may be displayed on display 206 during the ablation step of the microwave ablation procedure. Screen 700 shows an indicator 702 that the system is now operating in the ablation step. Screen 700 further shows a representation 704 of the surgical tool currently being used during the procedure, in the example ablation probe 130, and the ablation zone 706 based on the configured power and size of ablation probe 130, as well as the dimensions 708 of the ablation zone and a distance from the distal end of ablation probe 130 to the edge of the ablation zone. Screen 700 also shows a progress indicator 710 representing the progress of the ongoing ablation relative to ablation probe 130 and projected ablation zone 706. Screen 700 further includes a button 712 allowing the clinician to select a desired ablation zone chart based on the anatomical location of ablation probe 130 and in vivo or ex vivo data. Ex vivo data includes data acquired during an open surgical procedure, while in vivo data includes data acquired during other surgical procedures, such as laparoscopic surgical procedures. Screen 700 also includes a button 714 allowing the clinician to select a power setting for ablation probe 130, and a button 716 allowing the clinician to increase or decrease the size of the ablation zone based on the selected ablation zone chart.

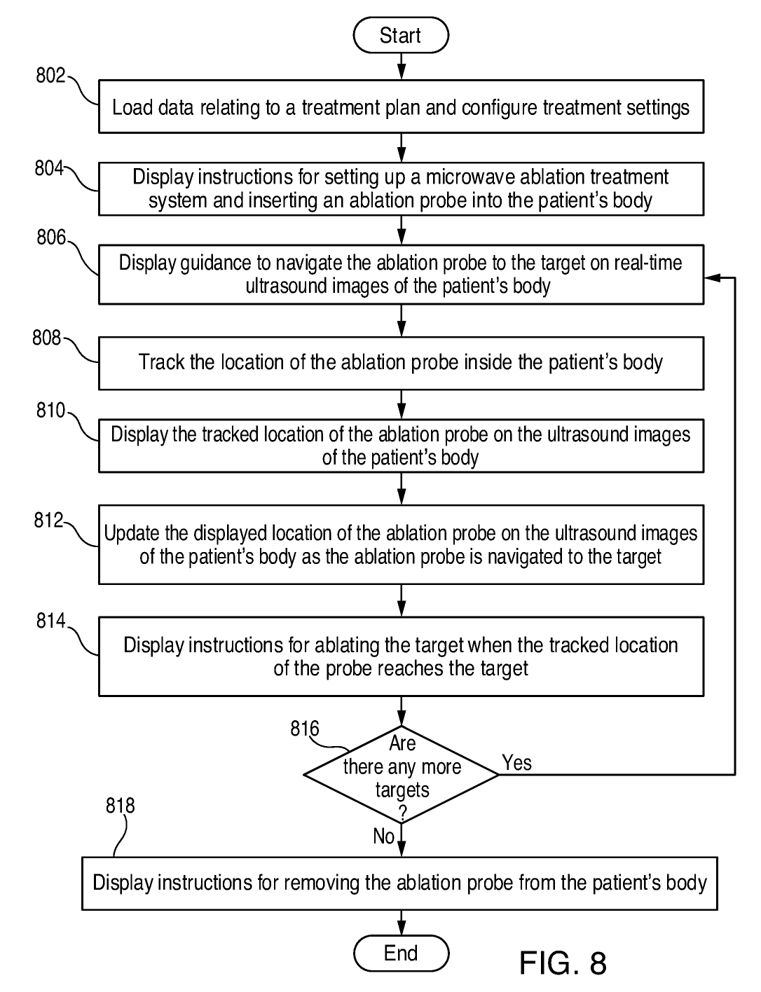

In some embodiments, system 10 may be operated without using the model generated during the planning phase of the microwave ablation treatment. In such embodiments, navigation of ablation probe 130 is guided by using ultrasound images, such as the ultrasound images generated by ultrasound sensor 140. During the guidance step of the microwave ablation procedure, the location of ablation probe 130 and the one or more targets are overlaid onto the ultrasound images generated by ultrasound sensor 140. By doing so, the location of ablation probe 130 may be viewed in relation to the ultrasound image plane to visualize a trajectory of ablation probe 130. The location of ablation probe 130 may be tracked by the EM tracking system, while the location of the one or more targets are determined based on data generated during the planning phase. A vector may also be displayed from the tip of ablation probe 130, showing the trajectory of ablation probe 130 and allowing the clinician to align ablation probe 130 to the target. An example method of performing a microwave ablation treatment procedure according to this embodiment is described below with reference to FIG. 8.

Referring now to FIG. 8, there is shown a flowchart of an example method for performing a microwave ablation procedure according to an embodiment of the present disclosure. At step 802, a clinician may use computing device 100 to load data relating to a treatment plan into application 216. The data may include the location of one or more targets within a patient's body, and a pathway to the one or more targets. The clinician may also configure the system settings for the microwave ablation procedure. For example, the clinician may preconfigure parameters related to the various tools to be used during the procedure, such as preconfiguring the output settings of ablation probe 130, such as a wattage, temperature, and/or duration of the ablation, for each target. By doing so, application 216 may automatically configure a different output of ablation probe 130 when ablation probe 130 reaches each target.

Then, at step 804, application 216, via user interface 218, displays instructions for setting up and configuring the microwave ablation system. Application 216 may also display instructions for inserting ablation probe 130 into the patient's body. Thereafter, at step 806, application 216 displays guidance to navigate ablation probe 130 to the target on ultrasound images generated by ultrasound sensor 140. The displayed guidance may include instructions for navigating ablation probe 130 to the one or more targets and/or a graphical map or pathway to the one or more targets that may be overlaid onto the ultrasound images.

The clinician then navigates ablation probe 130 to the target. While ablation probe 130 is navigated, application 216, at step 808, tracks the location of ablation probe 130 inside the patient's body, and, at step 810, displays the tracked location of ablation probe 130 on the ultrasound images of the patient's body generated by ultrasound sensor 140. Application 216, at step 812, iteratively updates the displayed location of ablation probe 130 on the ultrasound images as ablation probe 130 is navigated to the target.

When application 216 detects that ablation probe 130 has reached the target, application 216, at step 814, displays instructions for ablating the target. Thereafter, at step 816, application 216 determines if there are any more targets in the treatment plan that have yet to be treated. If the determination is yes, the process returns to step 806 where the guidance is updated to guide ablation probe 130 to the next target. If the determination is no, application 216, at step 818, displays instructions for removing ablation probe 130 from the patient's body.

In other embodiments, computing device 100 may be operated independently to control an electrosurgical generator. For example, as shown in FIG. 7, computing device 100 may display a control screen that enables a clinician to control an electrosurgical generator without interacting directly with the electrosurgical generator. The clinician may use computing device 100 to configure settings for the microwave ablation procedure. For example, the clinician may preconfigure output wattages and ablation zones for each target to be ablated during the procedure, as well as other settings related to the operation of the electrosurgical generator during the procedure.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. A method comprising:
displaying a three-dimensional model of at least a part of a patient's body generated based on image data acquired during imaging of the patient's body;
displaying a pathway for navigating an ablation probe to at least one ablation target within the patient's body;
tracking the location of the ablation probe inside the patient's body while the ablation probe is navigated along the pathway;
displaying the tracked location of the ablation probe on the three-dimensional model;
iteratively updating the displayed location of the ablation probe as the location of the ablation probe is tracked while the ablation probe is navigated inside the patient's body; and
ablating the at least one target when the ablation probe is navigated proximate to the at least one target.

2. The method according to claim 1, wherein the pathway to the at least one target is a straight line.

3. The method according to claim 1, wherein the pathway extends between the at least one target and the exterior of the body of the patient.

4. The method according to claim 1, further comprising tracking the location of at least one electromagnetic sensor located on the ablation probe.

5. The method according to claim 1, further comprising tracking the location of an ultrasound imager.

6. The method according to claim 5, further comprising displaying the tracked location of the ablation probe on real-time ultrasound images generated by the ultrasound imager.

7. The method according to claim 6, wherein the location of the ablation probe in relation to the at least one target is displayed on the real-time ultrasound images based on the tracked location of the ablation probe inside the patient's body.

8. The method according to claim 6, wherein the location of the ablation probe in relation to the at least one target is displayed on the real-time ultrasound images based on the tracked location of the ultrasound imager.

9. The method according to claim 1, further comprising displaying a model of a planned ablation zone in relation to the at least one target on the three-dimensional model.

10. The method according to claim 6, further comprising displaying a model of a planned ablation zone in relation to the at least one target on the real-time ultrasound images.

11. The method according to claim 6, further comprising displaying, on the real-time ultrasound images, a projected ablation zone relative to the ablation probe.

12. The method according to claim 1, further comprising displaying, on the three-dimensional model, a projected ablation zone relative to the ablation probe.

13. The method according to claim 1, wherein the displayed location of the ablation probe is iteratively updated in relation to the pathway as the location of the ablation probe is tracked while the ablation probe is navigated inside the patient's body.

14. The method according to claim 1, further comprising displaying, on the three-dimensional model, a vector from the tip of the ablation probe indicating the trajectory of the ablation probe.

15. The method according to claim 6, further comprising displaying, on the real-time ultrasound images, a vector from the tip of the ablation probe indicating the trajectory of the ablation probe.

16. The method according to claim 15, further comprising displaying, on the real-time ultrasound images, a shadow bar overlay indicating whether the trajectory of the ablation probe is in front of or behind the plane of the real-time ultrasound images.

17. The method according to claim 1, further comprising inserting the ablation probe percutaneously into the patient's body.
